(19) United States
(12) Patent Application Publication
(10) Pub. No.: US 2016/0228033 A1
(43) Pub. Date: Aug. 11, 2016

(54) REFERENCE DEVICE FOR SURGICAL NAVIGATION SYSTEM

(71) Applicant: IZI MEDICAL PRODUCTS, LLC, Owings Mills, MD (US)

(72) Inventor: HOLGER-CLAUS ROSSNER, FELDKIRCHEN B. MUENCHEN (DE)

(21) Appl. No.: 15/098,676

(22) Filed: Apr. 14, 2016

Related U.S. Application Data
(62) Division of application No. 14/245,170, filed on Apr. 4, 2014.

(51) Int. Cl. A61B 5/06

(52) U.S. Cl. CPC A61B 5/064; A61B 2090/3983

(57) ABSTRACT

A device and method for a surgical navigation system comprising a connection unit, a marker carrier unit removably attached to the connection unit, an attachment unit connected to the connection unit for fixing the device to a body part of a patient. The connection unit comprises an articulated arm and the marker carrier unit comprises an attachment area for removably attaching the marker carrier element to the connection unit.

### BACKGROUND

1. Field of the Invention

The present invention relates generally to surgical navigation systems. More particularly, the present invention relates to a referencing device for a surgical navigation system, a marker carrier unit for use in a referencing device as well as a method for surgical navigation.

2. Related Art

Surgical navigation systems are employed in a variety of surgical applications, for example, in neurosurgery, oral, maxillofacial and facial surgery, ear nose and throat (ENT) surgery or also for limb implantation in orthopedic surgery. Based on three-dimensional patient image data, which are obtained by means of X-ray images, computer tomography (CT), magnetic resonance tomography (MRT) and/or positron emission tomography (PET), surgical navigation systems of this type enable the position of medical instruments to be visualized in real-time in the patient image data in order to thereby assist the surgeon during operable procedures.

To this end, it may be necessary to record and monitor the position and orientation of the patient or a specific body part on which a surgical procedure is to be carried out—also referred to as “tracking.” Conventional referencing devices have been used usually comprising reference frames to which marking elements such as light-reflecting, spherical marker elements are attached. The light-reflecting spherical marker elements allow a stereo camera system of the navigation system to record the precise position and orientation of the referencing device.

Conventional navigation systems and/or referencing devices are known, for example, from documents DE 10 2011 054 730 A1, DE 698 33 881 T2, DE 10 2010 060 914 A1 or DE 60 2004 004 158 T2. WO 2006/012491 discloses marker elements together with a unit carrying the marker elements—referred to as reference frames—as a single disposable unit which can be produced by injection molding. However, traditional navigation systems do not always allow for the desired positioning and orientation of the referencing device, for example, due to structural limitations in the design of its arranged configuration and/or restrictions in movement such as limited multiple ranges of motion and/or operating degrees of freedom.

Another concern may include operating and maintaining a sterile environment during surgical procedures. Medical devices, such as referencing devices must also be sterile. Within such an environment, marker elements may be removably attached, for example, by means of a standardized clip attachment to pins arranged on the referencing device. The referencing device may thus be sterilized without marker elements and new, sterile, disposable marker elements may be utilized for each use. Conventional corresponding marker elements are known, for example, from document DE 10 2009 019 986 A1.

In order to deduce the position and orientation of a patient (or as the case may be, the body part of a patient on which a surgical procedure is to take place), and in order to produce a correct reference to the 3D image data, it is necessary to calibrate the surgical navigation system by executing a registration step. Various reference points are thereby successively localized on the patient using a navigation apparatus and correlated with corresponding points in the 3D image data.

The registration process determines the geometric relationship between the anatomic structures of interest and the 3-dimensional (3D) computer image constructed, for example, from the preoperative CT scan. Registration involves two steps. First, the reference sensor is secured to a non-mobile structure. Then, a registration tip, for example, is used sequentially to touch pre-selected registration points (e.g., fiducial markers). Registration points may be any anatomic structures that are recognizable on the preoperative image (e.g. teeth, skin, bone). Each time a registration point is touched with the registration tip, the computer records the location of the position sensor and the reference sensor. Using, for example, at least three registration points, the computer calculates the physical position of the anatomic structure with respect to the reference sensors. The computer then uses this registration information to measure the position of the pencil relative to the preoperative CT scan. The patient's body part can be mobilized freely without the need to re-initialize the registration process, because the reference sensor is rigidly attached to the relevant structure of the patient. By way of this registration, a correct, spatial reference between the 3D image data and the position and orientation of the body part of the patient can be produced.

In particular, in the case of surgical procedures involving the brain, it is usually not possible to simply be limited to reference points in the operating area for a necessarily precise registration, but rather it is necessary, in the vast majority of cases, to select a plurality of reference points at different locations on the body of the patient. Since for this purpose unhindered access to these locations on the body of the patient is necessary, registration must thus take place before the patient can be finally prepared for the actual surgical procedure and covered in a sterile manner in the areas outside of the operating area.

As a practical matter, and as it pertains to the registration device itself, following a successful registration procedure necessarily means he registration device must be considered as being potentially contaminated. Thus, appropriate measures for protecting the patient must be taken before the image-guided surgical procedure using the navigation system can take place. As such, the reference frame is thus usually detached from the fixation unit, sterilized, and provided with new sterile marker elements and reconnected to the fixation unit. The fixation unit as well as the interface between the fixation unit and the reference frame must next be draped and/or otherwise covered. To achieve this, holes are typically generated in medical drapes in order to allow the reference frame or its components to protrude therethrough and to subsequently attach to the fixation unit. Additional care to secure and maintain medical drapes is also provided in order to achieve a covering considered at least sufficiently secure. From a user perspective, this approach is presented as less than desirable since, on the one hand, the effort is labor intensive and significant staff effort is required in order to provide the necessary draping and covering for operational procedure. And, on the other hand, the draping and covering is often regarding as insufficiently secure for operating procedures. This risks the sterility of the operating environment and loss of time in addressing the same.

It is, therefore, an object of the present invention to overcome the deficiencies of the prior art to provide an improved apparatus capable of providing increased range of motion in at least multiple to an infinite amount of directions while more easily achieving and maintaining a sterile operating environment. It is a further goal of the present invention to provide a method and apparatus that achieves and maintains a dependable fixed position of the referencing device during operational procedures that eliminates the need to recalibrate the system.

### SUMMARY

The foregoing needs are met, to a great extent, by the present invention, wherein in one aspect a device is provided that in some embodiments comprises a connection unit, a marker carrier unit removably attached to the connection unit and an attachment unit connected to the connection unit for fixing the device to a body part of a patient. The connection unit comprises an articulated arm wherein the marker carrier unit comprises an attachment area for removably attaching the marker carrier element to the connection unit.

In accordance with another embodiment of the present invention, a method is provided that in some embodiments comprises removably attaching a marker carrier unit to a connection unit of a referencing device by connecting an end of the connection unit to an attachment area of the marker carrier unit. The method may also include fixing another end of the connection unit to a body part of a patient, registering a correct spatial reference between a 3D image data and a position and orientation of a body part of the patient and removing the marker carrier unit from the connection unit at a location of the attachment area. Embodiments of the disclosed method may also include draping the connection unit with a medical drape and removably attaching a sterile marker carrier unit to the connection unit by connecting an end of the connection unit to an attachment area of the sterile marker carrier unit such that the medical drape is disposed within the attachment area of the sterile marker carrier unit and secured between the sterile marker carrier unit and the connection unit.

In accordance with yet another embodiment of the present invention, a method is provided that in some embodiments comprises removably attaching a marker carrier unit to an attachment mechanism of a connection unit by inserting the attachment mechanism into an attachment area of the marker carrier unit, fixing another end of the connection unit to a body part of a patient and registering a correct spatial reference between a 3D image data and a position and orientation of a body part of the patient. The method may also include removing the marker carrier unit from the connection unit by detaching the attachment mechanism from the attachment area.

There has thus been outlined, rather broadly, certain embodiments of the invention in order that the detailed description of the invention herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as in the abstract, are for the purpose of description and should not be regarded as limiting.

As such, those skilled in the art will appreciate that the concept upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Still other aspects, features and advantages of the present invention are readily apparent from the following detailed description, simply by illustrating a number of exemplary embodiments and implementations, including the best mode contemplated for carrying out the present invention. The present invention also is capable of other and different embodiments, and its several details can be modified in various respects, all without departing from the spirit and scope of the present invention. Accordingly, the drawings and descriptions are to be regarded as illustrative in nature, and not as restrictive.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a perspective view of a referencing device for a surgical navigation system according to an embodiment of the present invention.

FIG. 2 is an exploded view of an exemplary ball and socket joint disposed near one end of an articulated arm of the referencing device according to one embodiment of the present invention.

FIG. 3 is an exploded view of an exemplary rotary joint disposed along an articulated arm of the referencing device according to one embodiment of the present invention.

FIG. 4 is side view depicting the assembly of an attachment foot in connection with an articulated arm being mated with a marker carrier unit according to one embodiment of the present invention.

FIG. 5 is an exploded view of another exemplary ball and socket joint disposed near another end of an articulated arm of the referencing device according to one embodiment of the present invention.

FIG. 6 is a top view of an exemplary design of an attachment foot mated in an exemplary recess of a marker carrier body according to one embodiment of the present invention.

FIG. 7 is a cross sectional view taken along C-C of FIG. 6 according to one embodiment of the present invention.

FIG. 8 is a top view of an exemplary design of an attachment foot mated in an exemplary recess of a marker carrier body according to one embodiment of the present invention.

FIG. 9 is a cross sectional view taken along D-D of FIG. 8 according to one embodiment of the present invention.

FIG. 10 is another side view of an assembly of an exemplary attachment foot being secured to an exemplary marker carrier body via an exemplary design of a clamp lever according to one embodiment of the present invention.

FIG. 11 is another side view of an assembly of an exemplary attachment foot being secured to an exemplary marker carrier body via an exemplary design of a clamp lever according to one embodiment of the present invention.

FIG. 12 is a partial view of an exemplary attachment foot attached to an articulated arm via a ball and socket joint according to an embodiment of the present invention.

FIG. 13 illustrates an embodiment of a marker carrier unit for assembly with the illustrated exemplary attachment foot according to an embodiment of the present invention.

FIG. 14 illustrates steps for preparing and affixing the disclosed referencing device onto a patient for a surgical navigation procedure according to an embodiment of the present invention.

### DETAILED DESCRIPTION OF THE PREFERRED EMBODIMENTS

### Definitions

Where the definition of terms departs from the commonly used meaning of the term, applicant intends to utilize the definitions provided below, unless specifically indicated.

For the purposes of the present invention, directional terms such as “top”, “bottom”, “upper”, “lower”, “above”, “below”, “left”, “right”, “horizontal”, “vertical”, “upward”, “downward”, etc., are merely used for convenience in describing the various embodiments of the present invention.

For purposes of the present invention, the term “ball and socket joint” (also referred to a ball joint) refers to a joint, as in a mechanical device, that permits rotary movement in all directions through the movement of a ball in a socket. The ball and socket joint is a joint in which the ball-shaped surface of one rounded member fits into a cup-like depression of another member. The distal member is capable of motion around an indefinite number of axes, which have one common center. It enables the member to move in many planes (almost all directions).

For purposes of the present invention, the term “distal” refers being situated away from a point of attachment or origin or a central point.

For purposes of the present invention, the term “drape” refers to the sterilized cloths that mark off an operative field. Typically the aforementioned cloths are arranged over a patient's body during an examination or treatment or during surgery and are designed to provide a sterile field around the area. “Draping” refers to the process thereof.

For purposes of the present invention, the term “Image-guided surgery” (IGS) refers to surgical procedures where the surgeon employs tracked surgical instruments in conjunction with preoperative or intraoperative images in order to indirectly guide the procedure. Image-guided surgery is part of the wider field of computer-assisted surgery. During a surgical procedure, the IGS tracks the probe position and displays the anatomy beneath it as, for example, three orthogonal image slices on a workstation-based 3D imaging system. Existing IGS systems use different tracking techniques including mechanical, optical, ultrasonic, and electromagnetic.

For purposes of the present invention, the term “indicia” refers distinctive marks, characteristic markers or indications.

For purposes of the present invention, the term “proximal” refers to being next to or nearest the point of attachment or origin, a central point, or the point of view; especially located toward the center of the body—compare distal. For purposes of the present invention, the term “distal” refers to the direction opposite the “proximal” direction.

For purposes of the present invention, the term “registering” refers to a process for determining the geometric relationship between an anatomic structure(s) of interest and a 3-dimensional (3D) computer image constructed, for example, from the preoperative CT scan. By way of this registration, a correct, spatial reference between the 3D image data and the position and orientation of the body part of the patient, observed by means of referencing device, can be produced.

For purposes of the present invention, the term “rotary joint” refers to a freely moving joint in which movement is limited to rotation; the rotary joint may be considered as a flexible joint that connects a stationary object with a rotating object in a piece of machinery, for example, factory and medical equipment.

For purposes of the present invention, the term “surgical navigation” refers to computer assisted surgery (CAS) representing a surgical concept and set of methods that use computer technology for pre-surgical planning and for guiding or performing surgical interventions. CAS is also known as computer aided surgery, computer assisted intervention, image guided surgery and surgical navigation.

For purposes of the present invention, the term “surgical navigation system” refers a system that allows visualization of an operative site and surgical instruments simultaneously and relates them to the patient's diagnostic images (e.g., computed tomographic (CT) scans and magnetic resonance imaging (MRI)). A surgical navigation system is used to guide the surgeon's movements during an operation. It may display the real-time position of each instrument and anatomical structure. These systems are used in orthopedics, ENT, neurology and other surgical specialties. Real-time observations occur via MRI, scanner, video camera or another imaging process. Navigation data are incorporated into the image to help the surgeon determine precise position within the organism. Medical imaging is sometimes used to plan an operation before surgery. Data integration enables the system to compare the actual position of the target object with the ideal location established during the planning phase. Such systems may be mechanical, electromagnetic or optical. The most common are optical devices, either passive or active. In the former, cameras locate specific markers such as reflective targets, particular shapes or colors. Active systems locate LEDs.

For purposes of the present invention, the term “x-direction” refers to the direction aligned with the x-axis of a coordinate system.

For purposes of the present invention, the term “y-direction” refers to the direction aligned with the y-axis of a coordinate system.

For purposes of the present invention, the term “z-direction” refers to the direction aligned with the z-axis of a coordinate system.

### Description

The invention will now be described with reference to the drawing figures, in which like reference numerals refer to like parts throughout. The following detailed description is of example embodiments of the presently claimed invention with references to the accompanying drawings. Such description is intended to be illustrative and not limiting with respect to the scope of the present invention. Such embodiments are described in sufficient detail to enable one of ordinary skill in the art to practice the subject invention, and it will be understood that other embodiments may be practiced with some variations without departing from the spirit or scope of the subject invention.

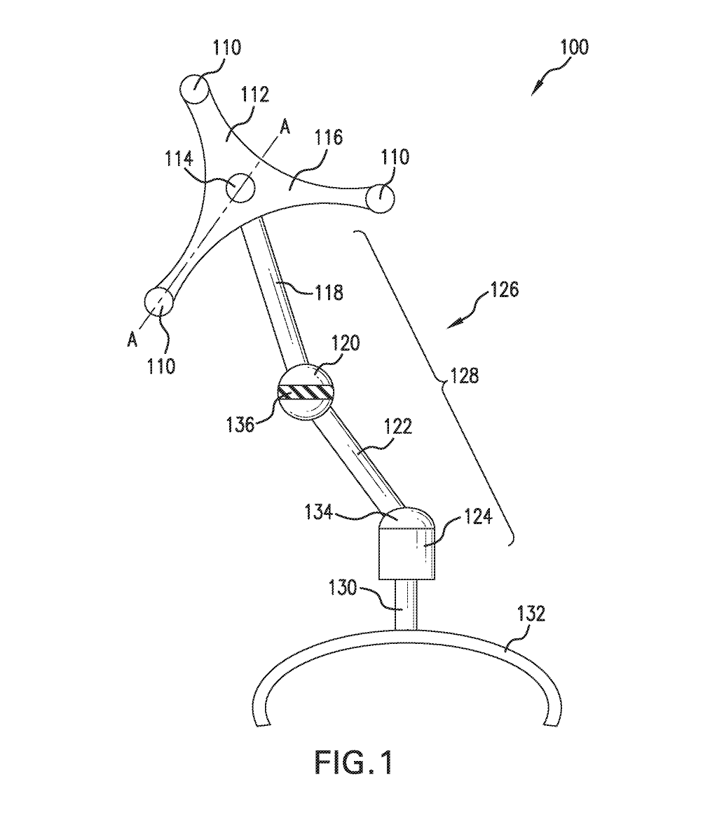

Turning to FIG. 1, a referencing device 100 is illustrated for positioning and mounting one or more marker elements 110 disposed on a marker carrier body 112 of a marker carrier unit 116. In the disclosed embodiment, marker carrier body 112 is substantially designed in as a three-armed unit wherein in the region of the end of each arm a marker element 110 is respectively arranged. Marker elements 110 may be designed as spherical marker elements including retro-reflective marker spheres, also referred to as passive reflective markers, and are widely used in image guidance systems. Embodiments of retro-reflective marker spheres may include those used to aid registration and instrument tracking during image guided surgery procedures such as neurological procedures, spine procedures and orthopedic procedures. Embodiments may include retro-reflective marker spheres having a high coefficient of retro-reflection on the external surface to provide feedback to the system/camera. Such surfaces may consist of micro glass spheres that reflect light. Depending on the medical application, different numbers and arrangements of retro-reflective marker spheres may be mounted on various types of surgical tools that may be used including that disclosed herein. Once mounted on a surgical probe, retro-reflective marker spheres provide an accuracy reference point for the surgical probe in three-dimensional space.

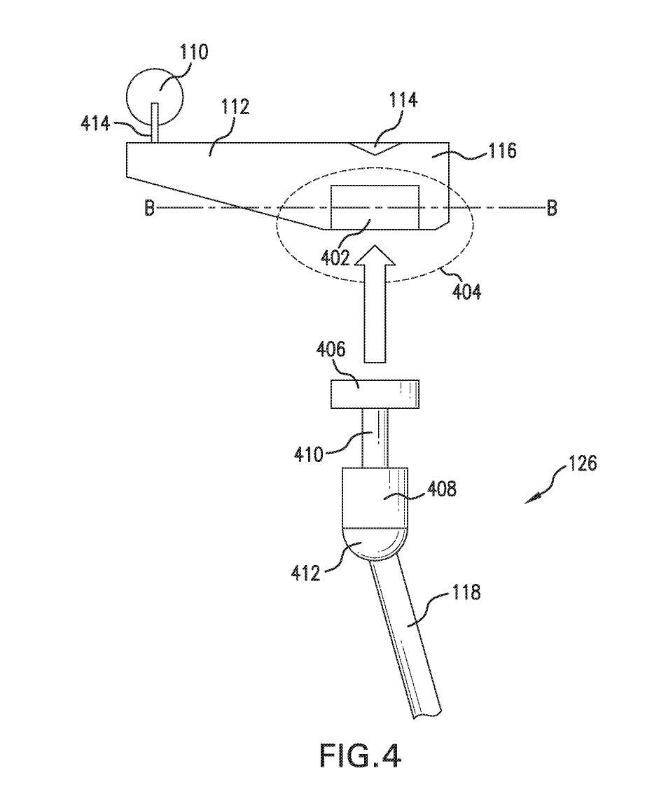

Disclosed embodiments provide the attachment of maker carrier unit 116 to a connection unit 126, for example, at a prescribed location 114 of marker carrier body 112. Prescribed location 114 may be located anywhere at the marker carrier unit 116. In one exemplary configuration, connection unit 126 includes an articulated arm 128 comprising a plurality of arm extensions 118, 122 and joints 408 (FIGS. 4), 120 and 124. While two arm extensions 118, 122 and three joints 408, 120 and 124 are described and illustrated in the exemplary drawings, it is readily appreciated that more arm extensions and joints may be employed by the disclosed invention, for example, to facility increased mobility and degrees of freedom in motion of articulated arm 128 and, hence, referencing device 100. Connection unit 126 may also comprise an attachment unit 132 and attachment foot 406 (FIG. 4), as described below.

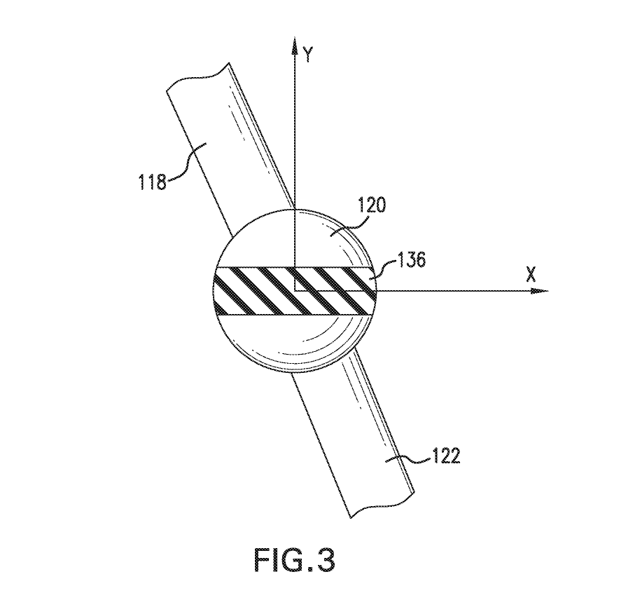

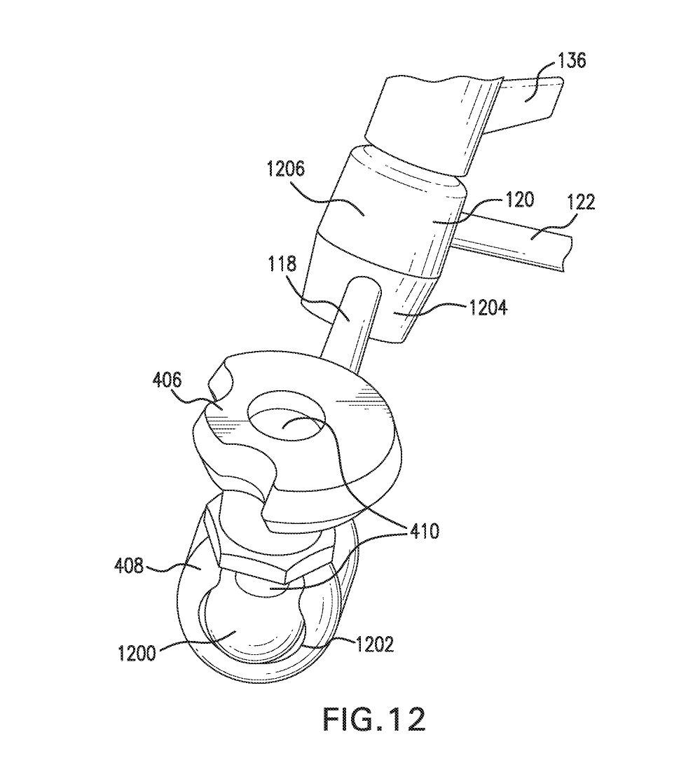

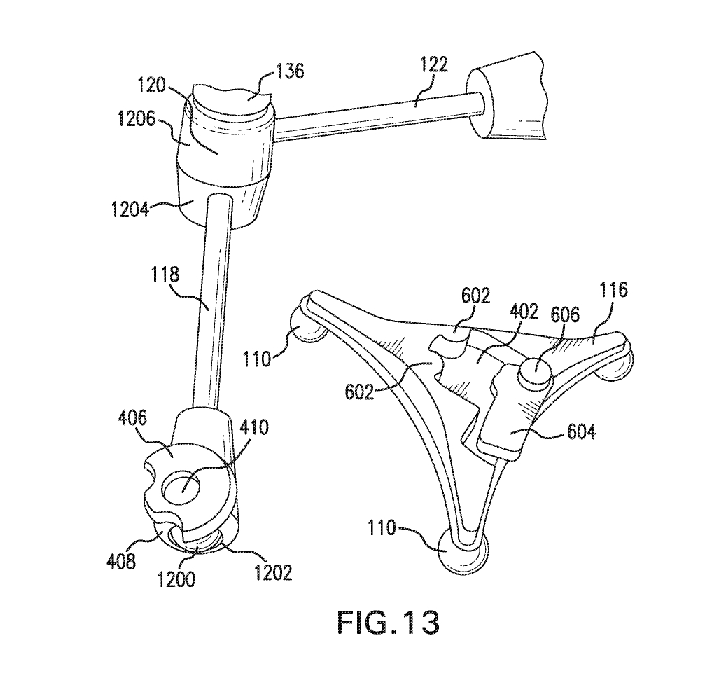

Arm extension 118 connects to marker carrier body 112 of marker carrier unit 116 at a first end and is configured to join with joint 120 at a second end. Arm extension 118 is a rigid arm extension and may be composed of stainless steel, medical grade steel, materials. In the disclosed embodiment, joint 120 may include a rotary joint for rotating arm extension 118 in the x-direction and y-direction direction (e.g., see FIG. 3). Turning to FIGS. 12 and 13, the exemplary configuration of joint 120 may include to segment portions, i.e., lower portion 1204 and upper portion 1206 forming a rotary joint. Lower portion 1204 moves relative to upper portion 1206 in a rotary fashion. Likewise, upper portion 1206 moves relative to lower portion 1204 in a rotary fashion. In the disclosed embodiment, arm extension 118 is rigidly connected to lower portion 1204 and arm extension 122 is rigidly connected to upper portion 1206 of joint 120. Thus each extension, 118, 122 moves relative to one another in rotary fashion.

Joint 120 may include a locking mechanism 136 for locking articulated arm 128 in a fixed position. For example, locking mechanism 136 may include a handle and a bolt (not shown) centering through joint 120 and in threaded engagement with the handle. Rotating the handle, for example, in a rotational direction, thereby, clamps down on joint 120 as the bolt is threaded into the handle thereby providing enough force to retain the joint and prevent further movement of the same. In the embodiment disclosed, for example, in FIGS. 12 and 13, upon tightening of the aforementioned handle, lower portion 1204 and upper portion 1206 would remain unmovable relative to one another and effectively “lock-down” joint 120 into a fixed or set configuration. Locking mechanism 136 may be configured to lock articulated arm 128, including joints 124, 408, such that articulated arm 128 becomes a completely rigid at a prescribed positioning.

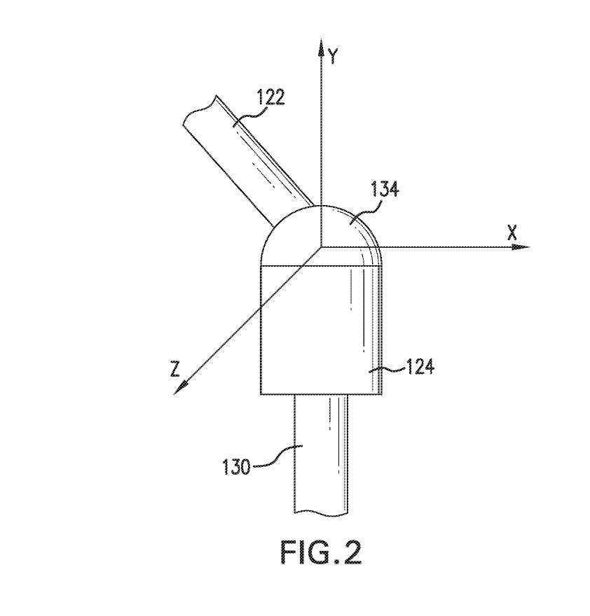

Arm extension 122 connects with joint 120 at a first end and is configured to connect with joint 124 at a second end. Thus, joint 120 forms a joint connection between arm extension 118 and arm extension 122. Joint 124 may include a ball joint wherein arm extension 122 connects with the ball portion of the pivot joint 134. The ball joint permits rotary movement of arm extension 122 in the x-direction, the y-direction, and z-direction (e.g., see FIG. 2). Arm extension 122 is a rigid arm extension that may be comprised of stainless steel, medical grade steel, materials.

An attachment unit 132 serves as a fixation device to connect to an extremity or body portion of a patient. Attachment unit 132 may also be configured to attach to other items used in surgery including, but not limited to, a human body part, a bone screw, or an implant. In the disclosed embodiment shown in FIG. 1, attachment unit 132 is configured as a head clamp designed to couple and attach with the head of a patient. However, it will be readily appreciated by those skilled in the art that attachment unit 132 may be designed to affix to other extremities or portions of a human patient including, for example, arms, legs, knees, angles, neck, wrists, hands, etc. Thus attachment unit 132 may comprise other alternative attachment mechanisms, for example, including attachment configurations for bone screws, spinal clamps, surgical pins, etc., or any other surgical mount suitable for affixing connection unit 126 to a human body part or extremity. The current embodiment depicts a mounting post 130 extending from attachment unit 132 and is configured to connectively attach to joint 124 thereby forming a joint connection between arm extension 122 and attachment unit 132. Thus, joint 124 permits movement of articulated arm 128 and marker carrier unit relative to attachment unit 132 which may be affixed at a prescribed location, for example, on the body of a patient. While a select number of joints have been illustrated in the drawings and described in the specification, more or less joints may be utilized to form the articulated arm of connection unit 126. In addition, other types of features may be utilized in the articulated arm including, for example, a telescopic feature employed in the arm extension for extending or shortening the arm extension along a length of connection unit 126.

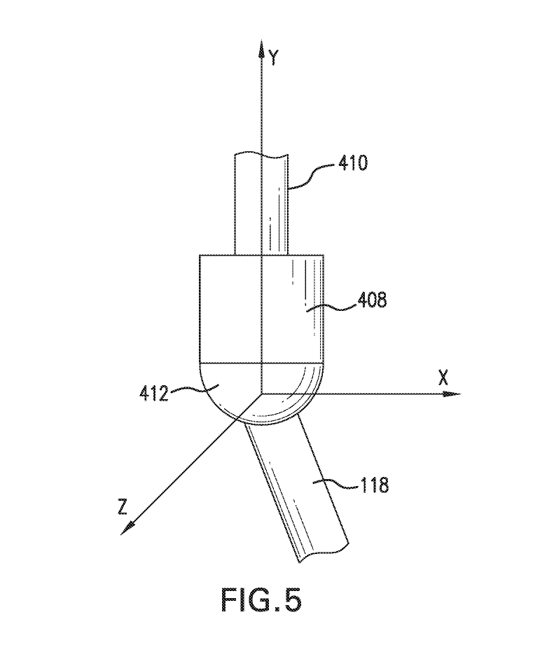

As previously mentioned above, arm extension 118 connects to marker carrier body 112 of marker carrier unit 116 at one end and is configured to join with joint 120 at another end. To connect with marker carrier unit 116, arm extension 118 connects with joint 408 thereby forming a joint connection therebetween. Turning to FIG. 4, joint 408 may comprise a ball joint wherein arm extension 118 connects with the ball portion of the pivot joint 412. Thus, the ball joint permits rotary movement of arm extension 118 in the x-direction, the y-direction, and z-direction (e.g., see FIG. 5). FIGS. 12 and 13 illustrate an embodiment of joint 408 represented as a ball and socket joint in which the ball-shaped surface of one rounded member 1200 fits into a cup-like depression of another member 1202. Coupled to joint 408 is an attachment mechanism for coupling arm extension 118 to marker carrier body 112 via joint 408. In the disclosed embodiment, the attachment mechanism comprises an attachment foot 406. Joint 408 permits attachment foot 406 to have a range of motion around an indefinite number of axes having a common center. The disclosed configuration enables attachment foot 406 to move in many planes (almost all directions) as further described below. Earlier described joint 124 may also include the ball joint configuration represented by joint 408 and illustrated, for example, in the exemplary embodiments of FIGS. 12 and 13.

Arm extension 118 is a rigid arm extension that may be comprised of stainless steel, medical grade steel, materials. As illustrated in FIG. 4, marker element 110 is mounted on mounting post 414 rigidly fixed to marker carrier body 112. Marker carrier body 112 comprises an attachment area 404 for receiving and coupling/mating an attachment mechanism, such as attachment foot 406, in receiving area 402. Receiving area 402 may be formed as a recess or cavity appropriately dimensioned and configured to receive and retain attachment foot 406 therein, as described below. Attachment foot 406 extends from joint 408 via mounting post 410. Marker carrier unit 116 is ultimately retained on connection unit 126 via the receipt and retention of attachment foot 406. Thus, by function of joint 124, connection unit 126 is permitted to rotate and pivot relative to attachment unit 132. By function of joint 408, marker carrier unit 116 is permitted to rotate and pivot relative to connection unit 126.

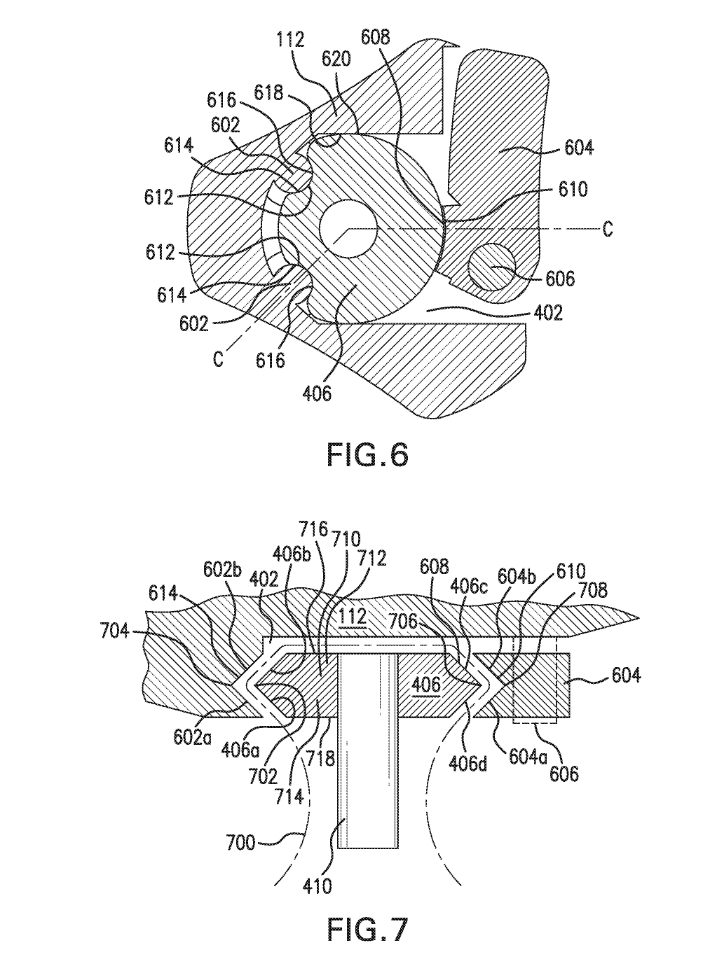

FIG. 6 illustrates a top view of one embodiment of attachment foot 406 mated in receiving area 402 of marker carrier body 112. When received within receiving area 402, select inner wall portions of marker carrier body 112 are sufficiently designed to contact points of the outer surface of attachment foot 406 to facilitate locating and securing the same therein. For example, in one disclosed embodiment locating contact surfaces 602 are formed to protrude into receiving area 402. Locating contact surfaces 602 act as an alignment mechanism of marker carrier body 112 for positioning onto attachment foot 406, as described below. Appropriately sized receiving areas 612 of attachment foot 406 are configured to receive corresponding locating contact surfaces 602 to form a mated configuration wherein the outer surface 614 of locating contact surfaces 602 generally abuts against the outer surface 616 of corresponding receiving areas 612. Side edge surface 618 of attachment foot 406 also generally abuts corresponding side edges 620 of receiving area 402.

A clamp lever 604 is provided to position and retain attachment foot 406 within receiving area 402. Pin 606 is disposed through clamp lever 604 such that clamp lever 604 pivots about pin 606. (As shown more easily in FIG. 7, pin 606 may be secured within the structure of marker carrier body 112.) When clamp lever 604 is pivoted about pin 606, the outer surface 610 of clamp lever 604 is rotated into contact with outer surface 608 of attachment foot 406 thereby providing a frictional interference fit in a clamped position. A material of attachment foot 406 and/or clamp lever 604 may be designed to withstand a certain amount of deflection to facilitate the frictional fit and retention of clamp lever 604 in the clamped position and thereby secure attachment foot 406 within receiving area 402.

FIG. 7 provides a cross sectional view taken along C-C of FIG. 6. In the exemplary embodiment, a medical drape 700 may be disposed over attachment foot 406 within receiving area 402. As illustrated in the current embodiment, the side circumference 710 of attachment foot 406 may be designed with a generally multi-angular configuration. Thus a top half 712 of the side circumference 710 may angle generally downwardly and away from a top surface 716 of attachment foot 406 to form a top half angled surface 406b. A bottom half 714 of the side circumference 710 may angle generally upwardly and away from a bottom surface 718 of attachment foot 406 to form a bottom half angled surface 406a. Top half angled surface 406b and bottom half angled surface 406a are configured to diverge into a point 702.

Outer surface 614 of protruding locating contact surfaces 602 is designed to mate in complimentary fashion with the design configuration of top half angled surface 406b and the bottom half angled surface 406a. Angled surfaces of outer surface 614 include a top half angled surface 602b and a bottom half angled surface 602a that diverge into point 704. Accordingly, top half angled surface 602b, bottom half angled surface 602a and point 704 of outside surface of contact surface 602 are formed in a complimentary configuration to mate with the angular design of corresponding top half angled surface 406b, bottom half angled surface 406a and point 702, respectively.

Next, the current embodiment of the configuration of outer surface 608 of attachment foot 406 with respect to outer surface 610 of clamp level 604 is described. The side circumference 710 of attachment foot 406 may be designed with a generally multi-angular configuration. Top half 712 of side circumference 710 may angle generally downwardly and away from top surface 716 of attachment foot 406 to form a top half angled surface 406c. Bottom half 714 of side circumference 710 may angle generally upwardly and away from a bottom surface 718 of attachment foot 406 to form a bottom half angled surface 406d. Top half angled surface 406c and bottom half angled surface 406d are configured to diverge at a point 706.

Outer surface 610 of clamp lever 604 is designed to mate in complimentary fashion with the design configuration of top half angled surface 406c and bottom half angled surface 406d. Angled surfaces of outer surface 610 include a top half angled surface 604b and a bottom half angled surface 604a that diverge at point 708. Accordingly, top half angled surface 604b, bottom half angled surface 604a of contact surface 610 and point 708 are formed in a complimentary configuration to mate with the angular design of corresponding top half angled surface 406c, bottom half angled surface 406d and point 706, respectively.

In operation, when clamp lever 604 is pivoted about pin 606 to bring outer surface 610 into contact with outer surface 608 of attachment foot 406, top half angled surface 406b, bottom half angled surface 406a and point 702 of attachment foot 406 mate with top half angled surface 602b, bottom half angled surface 602a and point 704 of outside surface 614 of locating contact surface 602, respectively. In this manner, locating contact surface 602 provides an alignment mechanism of marker carrier body 112 of the disclosed invention. This ensures that any marker carrier body 112 employing the designed receiving area 402 and the locating contact surfaces 602 will always be in the same position, location and/or orientation when mounted on the disclosed attachment foot 406 having corresponding complimentary receiving areas 612 after articulated arm 128 is set into a final position. Likewise, top half angled surface 604b, bottom half angled surface 604a and point 708 of contact surface 610 mate with the angular design of corresponding top half angled surface 406c, bottom half angled surface 406d and point 706 of attachment foot 406, respectively.

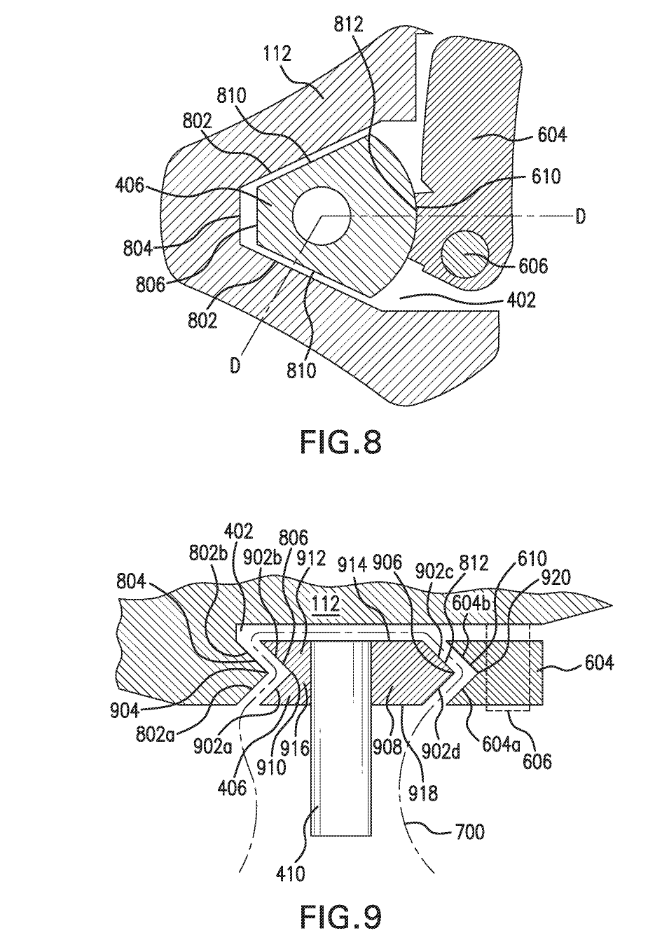

Turning to FIG. 8, another embodiment of attachment foot 406 mated in receiving area 402 of an exemplary marker carrier body 112 is shown. Attachment foot 406 is mated in receiving area 402 of marker carrier body 112. When received within receiving area 402, select inner wall portions of marker carrier body 112 are sufficiently designed to contact points of the outer surface of attachment foot 406 to facilitate locating and securing the same therein. For example, general angular side contact surfaces 802 are formed at a complimentary angle to side angular contact surfaces 810 of attachment foot 406. Angular side contact surfaces 802 are connected via a forward front surface 804. Forward front surface 804 corresponds to a complimentary forward surface 806 of attachment foot 406. Angular side contact surfaces 802 and forward front surface 804 act as an alignment mechanism of marker carrier unit 112. Thus, when clamp lever 604 is rotated about pin 606, outer surface 610 of clamp lever 604 is urged against rearward surface 812 of attachment foot 406. This motion urges forward surface 806 of attachment foot 406 into contact with forward front surface 804. Additionally, side angular contact surfaces 810 of attachment foot 406 abut angular side contact surfaces 802 of marker carrier body 112.

In the cross sectional view of FIG. 9 a medical drape 700 is disposed over attachment foot 406 within receiving area 402. As illustrated in the current embodiment, the side circumference 908 of attachment foot 406 may be designed with a generally angular configuration. Thus, at a location disposed near forward surface 806, a top half 912 of side circumference 908 may angle generally downwardly and inwardly from a top surface 914 of attachment foot 406 to form a top half angled surface 902b near forward surface 806. A bottom half 916 of the side circumference 908 may angle generally upwardly and inwardly from a bottom surface 918 of attachment foot 406 to form a bottom half angled surface 902a near forward surface 806. Top half angled surface 902b and bottom half angled surface 902a near forward surface 806 are configured to diverge into a point 910.

Forward front surface 804 acts as an abutment surface and is designed to mate in complimentary fashion with the configuration of top half angled surface 902b and bottom half angled surface 902a of attachment foot 406. Angled surfaces of forward front surface 804 include a top half angled surface 802b and a bottom half angled surface 802a that diverge into point 904. Accordingly, top half angled surface 802b, bottom half angled surface 802a and point 904 of forward front surface 804 are formed in a complimentary configuration to mate with the angular design of corresponding top half angled surface 902b, bottom half angled surface 902a and point 910, respectively.

Next, the current embodiment of the configuration of rearward surface 812 of attachment foot 406 with respect to outer surface 610 of clamp level 604 is described. The side circumference 908 of attachment foot 406 may be designed with a generally angular configuration. Top half 912 of side circumference 908 may angle generally downwardly and away from top surface 914 of attachment foot 406 to form a top half angled surface 902c. Bottom half 916 of side circumference 908 may angle generally upwardly and away from a bottom surface 918 of attachment foot 406 to form a bottom half angled surface 902d. Top half angled surface 902c and bottom half angled surface 902d are configured to diverge at a point 906.

Outer surface 610 of clamp lever 604 is designed to mate in complimentary fashion with the design configuration of top half angled surface 902c and bottom half angled surface 902d. Angled surfaces of outer surface 610 include a top half angled surface 604b and a bottom half angled surface 604a that diverge at point 920. Accordingly, top half angled surface 604b, bottom half angled surface 604a and point 920 of contact surface 610 are formed in a complimentary configuration to mate with the angular design of corresponding top half angled surface 902c, bottom half angled surface 902d and point 906, respectively.

In operation, when clamp lever 604 is pivoted about pin 606 to bring outer surface 610 into contact with rearward surface 812 of attachment foot 406, top half angled surface 902b, bottom half angled surface 902a and point 910 of attachment foot 406 mate with top half angled surface 802b, bottom half angled surface 802a and point 904 of forward front surface 804, respectively. In this manner, forward front contact surface 804 and in combination with side contact surfaces 802 provide an alignment mechanism of marker carrier body 112 of the disclosed invention. This ensures that any marker carrier body 112 employing the designed receiving area 402 and front contact surface 804 in combination with side contact surfaces 802 will always be in the same position, location and/or orientation when mounted on the disclosed attachment foot 406 having complimentary forward surface 806 and angular contact surfaces 810 after articulated arm 128 is set into a final position. Likewise, top half angled surface 604b, bottom half angled surface 604a and point 920 of contact surface 610 mate with the angular design of corresponding top half angled surface 902c, bottom half angled surface 902d and point 906 of attachment foot 406, respectively.

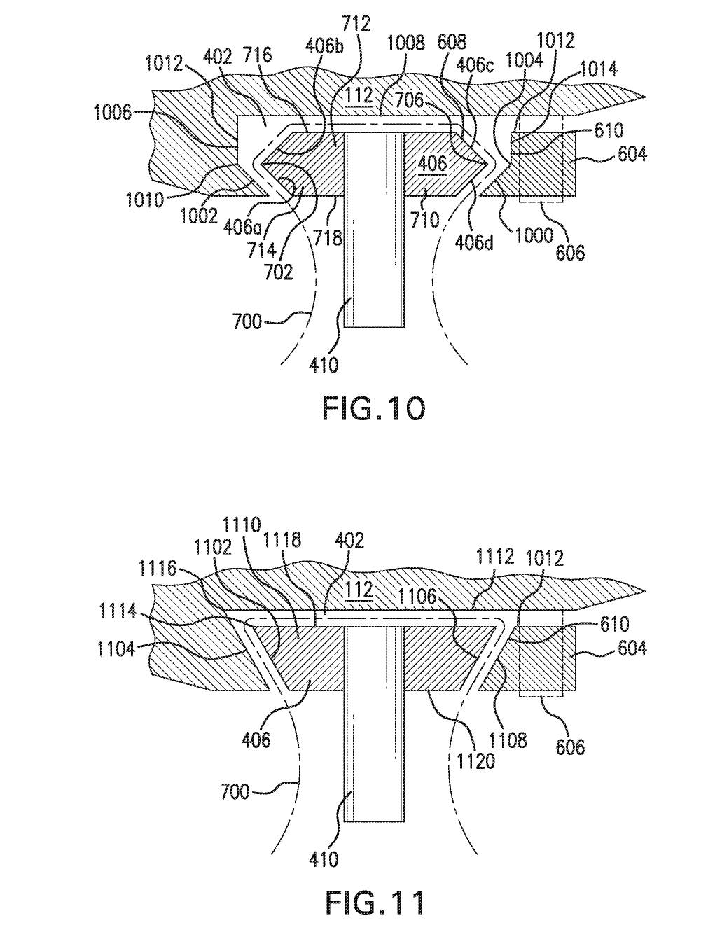

FIGS. 10 and 11 illustrate alternate embodiments of the attachment foot 406 and clamp lever 604 design. Turning to FIG. 10, a cross-sectional view of attachment foot 406 is disposed within receiving area 402. A medical drape 700 may be disposed over attachment foot 406 within receiving area 402. The side circumference 710 of attachment foot 406 may be designed with a generally multi-angular configuration. Thus a top half 712 of the side circumference 710 may angle generally downwardly and away from a top surface 716 of attachment foot 406 to form a top half angled surface 406b. A bottom half 714 of the side circumference 710 may angle generally upwardly and away from a bottom surface 718 of attachment foot 406 to form a bottom half angled surface 406a. Top half angled surface 406b and bottom half angled surface 406a are configured to diverge into a point 702.

The side wall surface 1012 of receiving area 402 is configured to mate with the surface of side circumference 710. An angular side surface 1002 of side wall surface 1012 is designed at a complimentary angle to mate in complimentary fashion with the bottom half angled surface 406a. Thus, surfaces of side wall surface 1012 include a wall 1006 extending downwardly and generally perpendicular from a top surface 1008 of receiving area 402. Side wall surface 1012 also includes angular side surface 1002 angled downwardly and inwardly from a point 1010 extending from a bottom of wall 1006. Accordingly, angular side surface 1002 and point 1010 of side wall surface 1012 are formed in a complimentary configuration to mate with the angular design of corresponding bottom half angled surface 406a and point 702, respectively.

Next, the current embodiment of the configuration of outer surface 608 of attachment foot 406 with respect to outer surface 610 of clamp level 604 is described. The side circumference 710 of attachment foot 406 may be designed with a generally multi-angular configuration. Top half 712 of side circumference 710 may angle generally downwardly and away from top surface 716 of attachment foot 406 to form a top half angled surface 406c. Bottom half 714 of side circumference 710 may angle generally upwardly and away from a bottom surface 718 of attachment foot 406 to form a bottom half angled surface 406d. Top half angled surface 406c and bottom half angled surface 406d are configured to diverge at a point 706.

Outer surface 610 of clamp lever 604 is designed to mate in complimentary fashion with the design configuration of bottom half angled surface 406d. Outer surface 610 includes a wall 1014 extending downwardly and generally perpendicular from a top surface 1012 of clamp lever 604. Outer surface 610 also includes angular side surface 1000 angled downwardly and inwardly from a point 1004 extending from a bottom of wall 1014. Accordingly, angular side surface 1000 and point 1004 of outer surface 610 are formed in a complimentary configuration to mate with the angular design of corresponding bottom half angled surface 406d and point 706, respectively.

In operation, when clamp lever 604 is pivoted about pin 606 to bring outer surface 610 into contact with outer surface 608 of attachment foot 406, bottom half angled surface 406a and point 702 of attachment foot 406 align with angular side surface 1002 and point 1010 of side wall surface 1012, respectively. Likewise, top half angled surface 604b, bottom half angled surface 604a and point 708 of contact surface 610 mate with the angular design of corresponding top half angled surface 406c, bottom half angled surface 406d and point 706 of attachment foot 406, respectively. In a final assembly, top surface 716 of attachment foot 406 may be abutted against top surface 1008 of receiving area 402 to secure attachment foot 406 within receiving area 402. In this manner, this ensures that any marker carrier body 112 employing the designed receiving area 402 of FIG. 10 will always be in the same position, location and/or orientation when mounted on the disclosed attachment foot 406 of FIG. 10 after articulated arm 128 is set into a final position.

Turning to an alternate embodiment depicted in FIG. 11, a cross-sectional view illustrates attachment foot 406 disposed within receiving area 402. A medical drape 700 is disposed over attachment foot 406 within receiving area 402. The side circumference 710 of attachment foot 406 may be designed with a generally angular configuration. A side profile of attachment foot 406 generally represents a trapezoidal shape wherein a top surface 1118 is slightly longer than a bottom surface 1120 of attachment foot 406. As shown, top surface 1118 is connected to bottom surface 1120 via a generally downwardly and inwardly angular wall surface 1102 disposed near angular side wall 1104 of receiving area 402. Point 1114 is formed at the joint wherein angular wall surface 1102 extends from top surface 1118. Angular side wall 1104 extends from a top surface 1112 of receiving area 402. Angular side wall 1104 may extend downwardly and inwardly at an angle complimentary to the angle formed by angular wall surface 1102. Point 1116 is formed at the joint wherein angular side wall 1104 extends from top surface 1112 of receiving area 402.

Likewise, top surface 1118 is connected to bottom surface 1120 via a generally downwardly and inwardly angular wall surface 1106 disposed near outer surface 610 of clamp lever 604. Outer surface 610 may form an angular surface 1108 generally complimentary to the angle formed by angular wall surface 1106. In operation, when clamp lever 604 is pivoted about pin 606 to bring angular surface 1108 into contact with angular wall surface 1106 of attachment foot 406, angular wall surface 1102 is brought into alignment with angular side wall 1104 and point 1114 meets with point 1116 in the aligned configuration. Additionally, angular surface 1108 is aligned with angular wall surface 1106, and top surface 1118 abuts top surface 1112 to retain attachment foot within fixed position of receiving area 402. In this manner, this ensures that any marker carrier body 112 employing the designed receiving area 402 of FIG. 11 will always be in the same position, location and/or orientation when mounted on the disclosed attachment foot 406 of FIG. 11 after articulated arm 128 is set into a final position.

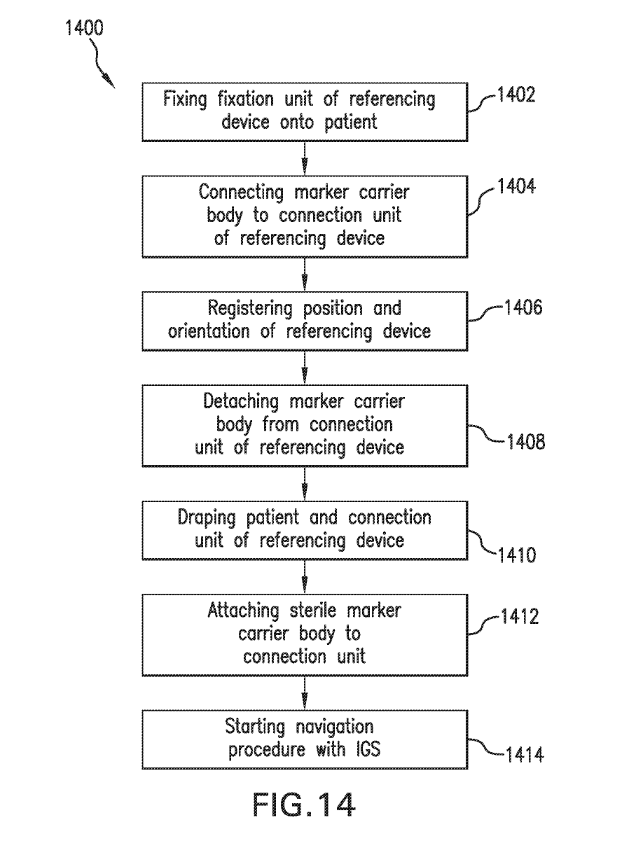

Given the improved features provided by embodiments of the disclosed referencing device 100, a method for preparing an image-guided, surgical navigation system is outlined herein. Turning to FIG. 14, a technique 1400 for preparing and utilizing an image-guided surgical navigation according to disclosed embodiments is depicted. Step 1402 requires fixing attachment unit 132 of referencing device 100 onto a patient. Step 1404 includes installing marker carrier body 112 onto connection unit 126. This may include adjusting components of connection unit 126 into a preferred position to orient marker carrier body 112 into a prescribed location, position and/or orientation. Adjustment of connection unit 126 may include manipulating arm extensions 118, 122 and joints 120, 124, 408, as necessary. Once an acceptable position is achieved, for example, an acceptable position of articulated arm 128 and orientation of marker carrier body 112, the articulated arm may be locked into a final position via locking mechanism 136 to secure the orientation of referencing device 100. Step 1406 includes registering a position and orientation of referencing device 100. This may include specifically registering a location, position and/or orientation of marker carrier unit 116. Having connection unit 126 locked into position, claim lever 604 may act as a detachment mechanism for releasing marker carrier body 112 from attachment foot 406. Accordingly, step 1408 includes detaching marker carrier body 112 from connection unit 126. Step 1410 provides draping the patient and connection unit 126 of referencing device 100. Disclosed embodiments provide that connection unit 126 includes articulated arm 126 including joint 408 and attachment foot 406. As illustrated in, at least, FIGS. 7, 9, 10, and 11, medical drape 700 is disposed over attachment foot 406 (and in a final assembly within receiving area 402). Step 1412 includes attaching a sterile marker carrier body 112 to connection unit 126. The sterile marker carrier body 112 may be the previous marker carrier body 112 which has since been sterilized, or it may be another sterile marker carrier body 112. A design of the disclosed marker carrier body 112 provides a uniquely configured receiving area that automatically locates, positions and orients marker carrier body 112 on a mountable attachment foot 406 in a complimentary mated fashion. The disclosed design consistently orients marker carrier body 112 to a repeatable prescribed position mounted on a complimentary configured attachment foot 406. The mounting and securing of marker carrier body 112 includes a feature of positioning and affixing medical draping in a secure and consistent manner Once a sterile marker carrier body 112 is fixed mounted to connection unit 126, step 1414 includes starting a navigation procedure via an image-guided surgery (IGS).

Having described the many embodiments of the present invention in detail, it will be apparent that modifications and variations are possible without departing from the scope of the present invention defined in the appended claims. For example, disclosed embodiments may provide certain indicia and/or colors on components of the disclosed invention such as, but not limited to, marker carrier unit 116, marker carrier body 112, and attachment foot 406. In one example, the aforementioned indicia and/or colors may correspond to a specific use or application associated with said indicia and/or colors. Such specific uses or applications associated with said indicia and/or colors may be employed, for example, in specific prescribed distinct surgical procedures or in certain environments or medical situations. These may include, but not limited to, for example, use in neuro and ENT surgery, spinal applications, soft/sensitive tissue applications and/or applying force applications. Furthermore, it should be appreciated that all examples in the present disclosure, while illustrating many embodiments of the present invention, are provided as non-limiting examples and are, therefore, not to be taken as limiting the various aspects so illustrated.

While the present invention has been disclosed with references to certain embodiments, numerous modifications, alterations, and changes to the described embodiments are possible without departing from the spirit and scope of the present invention, as defined in the appended claims. Accordingly, it is intended that the present invention not be limited to the described embodiments, but that it has the full scope defined by the language of the following claims, and equivalents thereof.

1.-36. (canceled)

37. A method of preparing a referencing device, comprising:
(a) removably attaching a marker carrier unit to an attachment mechanism of a connection unit by inserting the attachment mechanism into an attachment area of the marker carrier unit;
(b) fixing another end of the connection unit to a body part of a patient;
(c) registering a correct spatial reference between a 3D image data and a position and orientation of a body part of the patient;
(d) removing the marker carrier unit from the connection unit by detaching the attachment mechanism from the attachment area.

38. The method of claim 37, wherein the attachment mechanism comprises an attachment foot configured to fit within the attachment area of the marker carrier unit wherein step (a) comprises locking the attachment foot into place within the attachment area of the marker carrier unit to secure a rigid position and orientation of the of the marker carrier unit in a final assembly.

39. The method of claim 38, wherein the marker carrier unit comprises an alignment mechanism disposed within the attachment area, and the attachment foot comprises receiving areas corresponding to the alignment mechanism of the marker carrier unit, wherein step (a) comprises aligning the alignment mechanism of the marker carrier unit with the receiving areas of the attachment foot to position and orient the marker carrier unit on the connection unit in a final assembly.

40. The method of claim 39, wherein the alignment mechanism of the marker carrier unit comprises locating contact surfaces formed to protrude into the attachment area of the marker carrier unit, wherein step (a) comprises aligning the locating contact surfaces of the marker carrier unit with corresponding receiving areas of the attachment foot to position and orient the marker carrier unit on the connection unit in a final assembly.

41. The method of claim 40, wherein the marker carrier unit comprises a clamp lever disposed near a position of the attachment area,
wherein step (a) comprises pivoting the clamp lever of the marker carrier unit to engage and lock the attachment foot within the attachment area of the marker carrier unit to thereby provide a secure connection to position and orient the marker carrier unit.

42. The method of claim 41, wherein a side circumference of the attachment foot matches a complimentary profile of the alignment mechanism of the marker carrier unit, wherein a side circumference of the attachment foot matches a complimentary profile of a surface of the clamp lever engaged with the attachment foot,
wherein step (a) comprises mating the side circumference of the attachment foot to the complimentary profile of the alignment mechanism of the marker carrier unit by pivoting the clamp lever to engage and lock the attachment foot.

43. The method of claim 42, wherein the side circumference includes a multi-angled design.

44. The method of claim 37, further comprising:
(e) draping the connection unit with a medical drape; and
removably attaching a sterile marker carrier unit to the connection unit by connecting the attachment mechanism of the connection unit to an attachment area of the sterile marker carrier unit such that the medical drape is disposed within the attachment area of the sterile marker carrier unit and secured between the sterile marker carrier unit and the connection unit.

45. The method of claim 44, wherein step (e) further comprises draping the attachment unit with the medical drape.
